(19) United States
(12) Patent Application Publication
(10) Pub. No.: US 2023/0201564 A1
(43) Pub. Date: Jun. 29, 2023

(54) RE-SEALABLE MEMBER AND NOSE MEMBER OF DISTAL BEARING SUPPORT

(71) Applicant: TC1 LLC, St. Paul, MN (US)

(72) Inventors: Michael R. Butler, Dublin, CA (US); Stephen D. Pacetti, San Jose, CA (US); Phyllis K. Yuen, Fremont, CA (US)

(21) Appl. No.: 18/069,939

(22) Filed: Dec. 21, 2022

Related U.S. Application Data
(60) Provisional application No. 63/294,223, filed on Dec. 28, 2021.

(51) Int. Cl. A61M 60/414; A61M 60/148

(52) U.S. Cl. CPC A61M 60/414; A61M 60/148

(57) ABSTRACT

In various embodiments, a catheter pump is disclosed herein. The catheter pump can include an elongated catheter body having a distal portion including an expandable cannula having an inlet and an outlet. An impeller assembly can include an impeller shaft and one or more blades. The impeller blades can draw blood into the expandable cannula when rotated. Further, an expandable support can have a mounting portion. The mounting portion can have a cylindrical member disposed on the impeller shaft and can include an enlarged distal portion at a distal end of the impeller shaft and a nose member. Further, a re-sealable member can be disposed in the enlarged distal portion of the cylindrical member and can have a path through the re-sealable member through which a guidewire can be positioned. Further, the nose member can have an aperture distal of the re-sealable member for passage of the guidewire.

### CROSS REFERENCE TO RELATED APPLICATIONS

This application claims the benefit of priority to U.S. Provisional Pat. Application No. 63/294,223, filed Dec. 28, 2021, the contents and disclosure of which are incorporated by reference herein in their entirety.

### BACKGROUND

This application is directed to pumps for mechanical circulatory support of a heart. In particular, this application is directed to a re-sealable member and a nose member of a support structure for an impeller assembly that can be used in a catheter pump.

Heart disease is a major health problem that has a high mortality rate. Physicians increasingly use mechanical circulatory support systems for treating heart failure. The treatment of acute heart failure requires a device that can provide support to the patient quickly. Physicians desire treatment options that can be deployed quickly and minimally-invasively.

Intra-aortic balloon pumps (IABP) are currently the most common type of circulatory support devices for treating acute heart failure. IABPs are commonly used to treat heart failure, such as to stabilize a patient after cardiogenic shock, during treatment of acute myocardial infarction (MI) or decompensated heart failure, or to support a patient during high risk percutaneous coronary intervention (PCI). Circulatory support systems may be used alone or with pharmacological treatment.

In a conventional approach, an IABP is positioned in the aorta and actuated in a counterpulsation fashion to provide partial support to the circulatory system. More recently, minimally-invasive rotary blood pumps have been developed in an attempt to increase the level of potential support (i.e. higher flow). A rotary blood pump is typically inserted into the body and connected to the cardiovascular system, for example, to the left ventricle and the ascending aorta to assist the pumping function of the heart. Other known applications include pumping venous blood from the right ventricle to the pulmonary artery for support of the right side of the heart. An aim of acute circulatory support devices is to reduce the load on the heart muscle for a period of time, to stabilize the patient prior to heart transplant, or for continuing support.

There is a need for improved mechanical circulatory support devices for treating acute heart failure. Fixed cross-section ventricular assist devices designed to provide near full heart flow rate are either too large to be advanced percutaneously (e.g., through the femoral artery without a cutdown) or provide insufficient flow.

There is a need for a pump with improved performance and clinical outcomes. There is a need for a pump that can provide elevated flow rates with reduced risk of hemolysis and thrombosis. There is a need for a pump that can be inserted minimally-invasively and provide sufficient flow rates for various indications while reducing the risk of major adverse events. In one aspect, there is a need for a heart pump that can be placed minimally-invasively, for example, through a 15FR or 12FR incision. In one aspect, there is a need for a heart pump that can provide an average flow rate of 4 Lpm or more during operation, for example, at 62 mmHg of head pressure. While the flow rate of a rotary pump can be increased by rotating the impeller faster, higher rotational speeds are known to increase the risk of hemolysis, which can lead to adverse outcomes and in some cases death. Accordingly, in one aspect, there is a need for a pump that can provide sufficient flow while minimizing the likelihood of hemolysis at high rotational speeds. These and other problems are overcome by the inventions described herein.

Further, there is a need for providing an operative device of the pump capable of pumping blood at high flow rates while reducing the risk of hemolysis at the operative device. For example, when an impeller assembly is provided at the operative device, the high rate of rotation of the impeller may cause hemolysis, as blood flows past the highspeed impeller. Accordingly, there is a need for reducing the risk of hemolysis at the operative device of the pump, particularly when movable components are disposed at the operative device.

### SUMMARY

There is an urgent need for a pumping device that can be inserted percutaneously and also provide full cardiac rate flows of the left, right, or both the left and right sides of the heart when called for.

In one embodiment, a catheter pump is disclosed. The catheter pump can include an elongated catheter body having a distal portion including an expandable cannula having an inlet and an outlet. The expandable cannula can have a delivery profile and an operational profile larger than the delivery profile. An impeller assembly can include an impeller shaft, and an impeller body can include one or more blades. The impeller blades can draw blood into the expandable cannula when rotated. Further, an expandable support can have a mounting portion disposed on the impeller shaft distal of the impeller body to maintain a position of the impeller assembly relative to a cannula wall. The mounting portion can have a cylindrical member disposed on the impeller shaft and can include an enlarged distal portion having an inner diameter greater than the enlarged diameter at a distal end of the impeller shaft and a nose member. Further, a re-sealable member can be disposed in the enlarged distal portion of the cylindrical member. The re-sealable member can have a path through the re-sealable member along a length dimension of the re-sealable member through which a guidewire can be positioned. Further, the nose member can be coupled with the distal portion of the cylindrical member and can include a distal portion with an aperture distal of the re-sealable member for passage of the guidewire. The re-sealable member can reseal along the path through the re-sealable member when the guidewire is removed. In some embodiments, the re-sealable member can be a septum of varying shapes, with varying path lengths through the re-sealable member. In other embodiments, the catheter pump can include a plurality of re-sealable members, including a distal re-sealable member disposed in the enlarged distal portion of the cylindrical member and a proximal re-sealable member disposed in a proximal portion of the elongated catheter body. In yet other embodiments, the re-sealable member can be an elastomer doughnut that includes a closed off hole. Further, in other embodiments, the elongated catheter body can include a proximal portion with an anti-foaming agent.

In another embodiment, an apparatus for inducing motion of a fluid relative to the apparatus is disclosed. The apparatus can comprise a motor. An elongated catheter body can be coupled with the motor. The elongated catheter body can include an expandable distal portion having an inlet and an outlet and a support structure disposed about a lumen. The expandable distal portion can have a delivery profile and an operational profile larger than the delivery profile. The apparatus can include an impeller comprising at least one impeller blade. The apparatus can further include an expandable impeller support having an arcuate outer surface in contact with the support structure at least when the expandable distal portion has the operational profile. The apparatus can further include a re-sealable member disposed distally of the impeller. The apparatus can further include a nose member disposed distally of the re-sealable member and can include a distal portion with an aperture distal of the re-sealable member for passage of the guidewire. Operation of the motor can cause rotation of the impeller to draw blood into the lumen. The re-sealable member can reseal along the path through the re-sealable member when the guidewire is removed. In some embodiments, the re-sealable member can be a septum of varying shapes, with varying path lengths through the re-sealable member. In other embodiments, the catheter pump can include a plurality of re-sealable members, including distal re-sealable member disposed in the enlarged distal portion of the cylindrical member and a proximal re-sealable member disposed in a proximal portion of the elongated catheter body. In yet other embodiments, the re-sealable member can be an elastomer doughnut that includes a closed off hole. Further, in other embodiments, the elongated catheter body can include a proximal portion with an anti-foaming agent.

### BRIEF DESCRIPTION OF THE DRAWINGS

A more complete appreciation of the subject matter of this application and the various advantages thereof can be realized by reference to the following detailed description, in which reference is made to the accompanying drawings in which:

FIG. 1 illustrates one embodiment of a catheter pump configured for percutaneous application and operation;

FIG. 2 is a plan view of one embodiment of a catheter assembly adapted to be used with the catheter pump of FIG. 1;

FIGS. 3A-3C illustrate the relative position of an impeller blade and an inner surface of an impeller housing in an undeflected configuration;

FIG. 4 shows the catheter assembly similar to that of FIG. 2 in position within the anatomy;

FIG. 5 shows a cross-sectional view of one embodiment of a re-sealable member and a nose member of a distal bearing support;

FIGS. 6A-6C show a top-view of different embodiments of an opening in a re-sealable member of a distal bearing support;

FIG. 7 shows a cross-sectional view of another embodiment of a re-sealable member and a nose member of a distal bearing support;

FIG. 8 shows a cross-sectional view of yet another embodiment of a re-sealable member and a nose member of a distal bearing support;

FIGS. 9A-9B show a cross-sectional view of embodiments of a re-sealable member of a distal bearing support;

FIG. 10 shows a cross-sectional view of another embodiment of a re-sealable member of a distal bearing support; and

FIG. 11 shows a cross-sectional view of yet another embodiment of a re-sealable member of a distal bearing support.

More detailed descriptions of various embodiments of components for heart pumps useful to treat patients experiencing cardiac stress, including acute heart failure, are set forth below.

### DETAILED DESCRIPTION

This application is directed to apparatuses for inducing motion of a fluid relative to the apparatus. In particular, the disclosed embodiments generally relate to various configurations for a re-sealable member disposed distally of an impeller and a nose member disposed distally of the re-sealable member as part of a percutaneous catheter pump. As discussed in greater detail below, a re-sealable member and nose member can be advantageous to reseal the percutaneous catheter pump following guidewire removal once the catheter pump is placed in a patient’s heart. For example, in the disclosed embodiments, the re-sealable member can be a septum, with a path through the re-sealable member along a length dimension of the re-sealable member through which a guidewire can be positioned. Further, the nose member can have a distal portion with an aperture distal of the re-sealable member for passage of the guidewire. The re-sealable member can be configured to seal when the guidewire is withdrawn from the pump. The re-sealable member as disclosed herein can act in various embodiments to seal the catheter pump once placed in the heart of a patient, facilitating the reduction of hemolysis at the operative device of the pump and the flow of pumped blood through the heart of the patient without leaks into the operative device of the pump.

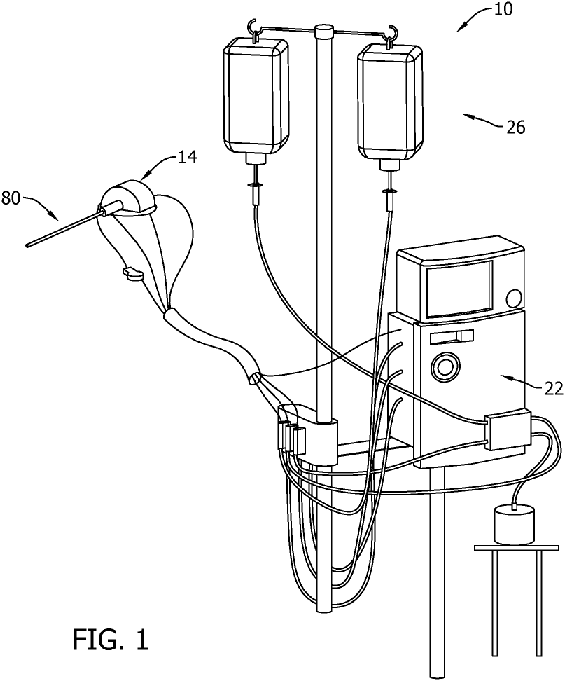

FIGS. 1-4 show aspects of one embodiment of a catheter pump that can provide high performance flow rates. FIG. 1 illustrates one embodiment of a catheter pump configured for percutaneous application and operation. The pump 10 includes a motor 14 driven by a controller 22. The controller 22 directs the operation of the motor 14 and an infusion system 26 that supplies a flow of infusate in the pump 10. A catheter system 80 that can be coupled with the motor 14 houses an impeller within a distal portion thereof. In various embodiments, the impeller is rotated by the motor 14 when the pump 10 is operating. For example, the motor 14 can be disposed outside the patient. In some embodiments, the motor 14 is separate from the controller 22, e.g., to be placed closer to the patient. In other embodiments, the motor 14 is part of the controller 22. In still other embodiments, the motor is miniaturized to be insertable into the patient. Such embodiments allow the drive shaft to be much shorter, e.g., shorter than the distance from the aortic valve to the aortic arch (about 5 cm or less).

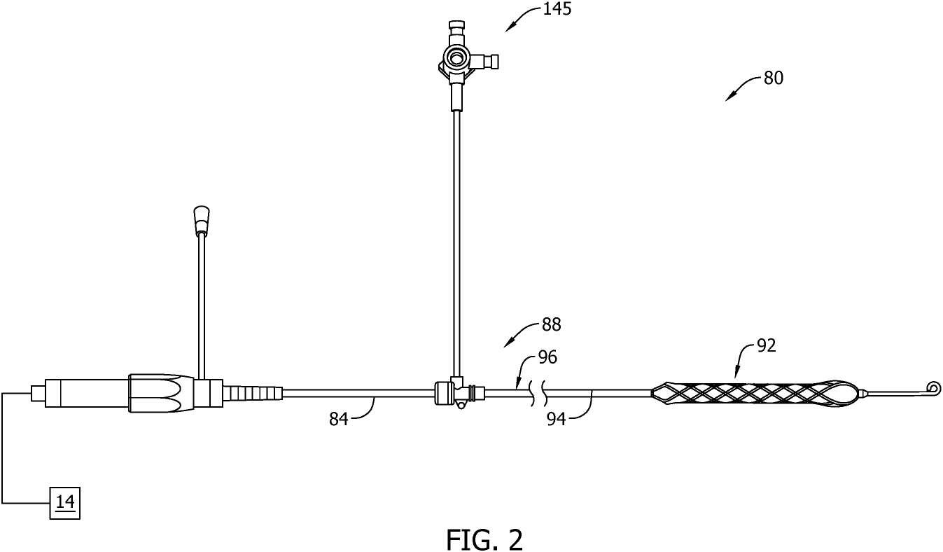

FIG. 2 shows features that facilitate small blood vessel percutaneous delivery and high performance, including up to and in some cases exceeding normal cardiac output in all phases of the cardiac cycle. In particular, the catheter system 80 includes a catheter body 84 and a sheath assembly 88. One embodiment of a blood flow assembly 92 is coupled with the distal end of the catheter body 84. At least a portion of the blood flow assembly 92 is expandable and collapsible. For example, the blood flow assembly 92 can include an expandable and collapsible cannula. The cannula can be formed of a superelastic material, and in some embodiments, may have various shape memory material properties. The blood flow assembly 92 also can include an expandable and collapsible impeller. The cannula and impeller are discussed more below. In the collapsed state, the distal end of the catheter system 80 can be advanced to the heart, for example, through an artery. In the expanded state, the blood flow assembly 92 is able to pump or output blood at high flow rates. FIGS. 2-4 illustrate the expanded state of one embodiment. The collapsed state can be provided by advancing a distal end 94 of an elongate body 96 of the sheath assembly 88 distally over the cannula of the blood flow assembly 92 to cause the blood flow assembly 92 to collapse. This provides an outer profile throughout the catheter system 80 that is of small diameter, for example a catheter size of about 12.5 Fr.

A proximal portion of the catheter body 84 may comprise a flow diverter chamber or an inner sheath. In some embodiments, the flow diverter chamber and/or the inner sheath may comprise an added anti-foaming agent to stop blood flow from entering the catheter. The anti-foaming agent may be oil-based, water-based, or silicone-based.

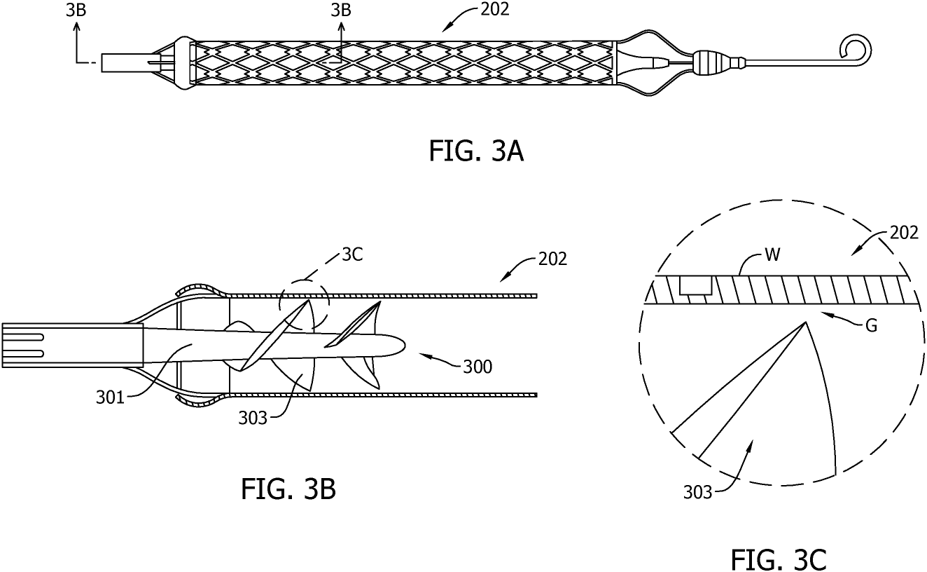

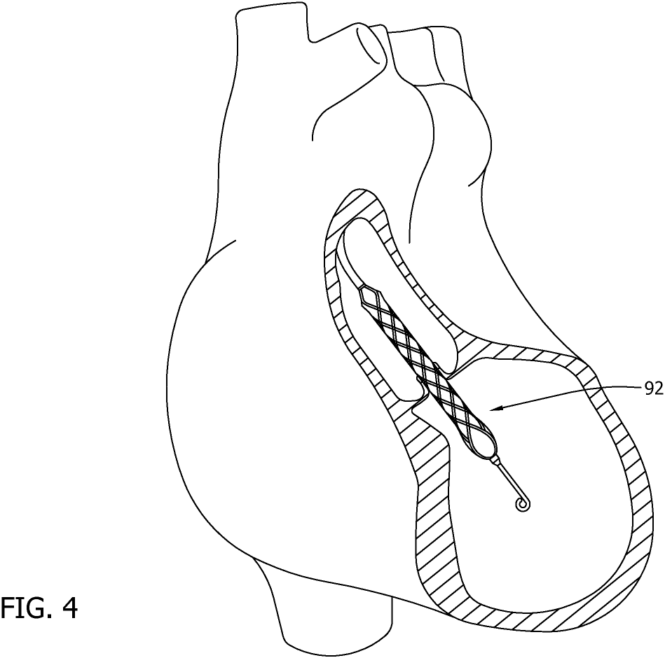

With reference to FIGS. 3A-3C, the operative device of the pump can include an impeller 300 having one or more blades 306. The one or more blades 306 can extend from an impeller hub 301. It can be desirable to increase the flow rate of the heart pump while ensuring that the impeller 300 can be effectively deployed within a subject. For example, an impeller can include one or more blades 306 that are configured to be inserted into a subject in a stored, or compressed, configuration. When the impeller 300 is positioned in the desired location, e.g., a chamber of a subject’s heart as shown in FIG. 4, the blades 306 of the impeller 300 can self-expand into a deployed or expanded configuration, in which the blades 306 extends radially from the impeller hub 301.

As shown in FIGS. 3A-3B, the impeller 300 can be positioned within a cannula or housing 202. A free end of the blades 306 can be separated from the wall W of the housing 202 by a tip gap G. The housing 202 can also have a stored, or compressed configuration, and a deployed or expanded configuration. The housing 202 and impeller 300 may deploy from the stored configurations from within the sheath assembly 88 into the expanded configuration. In such implementations, the sheath assembly 88 can keep the blades 306 and the housing 202 compressed until the blades 306 and housing 202 are urged from within a lumen of the sheath assembly 88. Once the blades 306 are released from the sheath assembly, the blades 306 can self-expand to a deployed configuration using strain energy stored in the blades 306 due to deformation of the blades 306 within the sheath assembly 88. The housing 202 may also self-deploy using stored strain energy after being urged from the sheath.

In the stored configuration, the impeller 300 and housing 202 have a diameter that is preferably small enough to be inserted percutaneously into a patient’s vascular system. Thus, it can be advantageous to fold the impeller 300 and housing 202 into a small enough stored configuration such that the housing 202 and impeller 300 can fit within the patient’s veins or arteries. In some embodiments, therefore, the impeller 300 can have a diameter in the stored configuration corresponding to a catheter size between about 8 Fr and about 21 Fr. In one implementation, the impeller 300 can have a diameter in the stored state corresponding to a catheter size of about 9 Fr. In other embodiments, the impeller 300 can have a diameter in the stored configuration between about 12. Fr and about 21 Fr. For example, in one embodiment, the impeller 300 can have a diameter in the stored configuration corresponding to a catheter size of about 12-12.5 Fr.

When the impeller 300 is positioned within a chamber of the heart, however, it can be advantageous to expand the impeller 300 to have a diameter as large as possible in the expanded or deployed configuration. In general, increased diameter of the impeller 300 can advantageously increase flow rate through the pump. In some implementations, the impeller 300 can have a diameter corresponding to a catheter size greater than about 12 Fr in the deployed configuration. In other embodiments, the impeller 300 can have a diameter corresponding to a catheter size greater than about 21 Fr in the deployed or expanded configuration.

In various embodiments, it can be important to increase the flow rate of the heart pump while ensuring that the operation of the pump does not harm the subject. For example, increased flow rate of the heart pump can advantageously yield better outcomes for a patient by improving the circulation of blood within the patient. Furthermore, the pump should avoid damaging the subject. For example, if the pump induces excessive shear stresses on the blood and fluid flowing through the pump (e.g., flowing through the cannula), then the impeller can cause damage to blood cells, e.g., hemolysis. If the impeller damages a large number of blood cells, then hemolysis can lead to negative outcomes for the subject. As will be explained below, various cannula and/or impeller parameters can affect the pump’s flow rate as well as conditions within the subject’s body.

When activated, the pump 10 can effectively increase the flow of blood out of the heart and through the patient’s vascular system. In various embodiments disclosed herein, the pump 10 can be configured to produce a maximum flow rate (e.g., low mm Hg) of greater than 4 Lpm, greater than 4.5 Lpm, greater than 5 Lpm, greater than 5.5 Lpm, greater than 6 Lpm, greater than 6.5 Lpm, greater than 7 Lpm, greater than 7.5 Lpm, greater than 8 Lpm, greater than 9 Lpm, or greater than 10 Lpm. In various embodiments, the pump can be configured to produce an average flow rate of greater than 2 Lpm, greater than 2.5 Lpm, greater than 3 Lpm, greater than 3.5 Lpm, greater than 4 Lpm, greater than 4.25 Lpm, greater than 4.5 Lpm, greater than 5 Lpm, greater than 5.5 Lpm, or greater than 6 Lpm.

FIG. 4 illustrates one use of the pump 10. A distal portion of the pump 10, which can include an blood flow assembly 92, is placed in the left ventricle (LV) of the heart to pump blood from the LV into the aorta. The pump 10 can be used in this way to treat patients with a wide range of conditions, including cardiogenic shock, myocardial infarction, and other cardiac conditions, and also to support a patient during a procedure such as percutaneous coronary intervention. One convenient manner of placement of the distal portion of the pump 10 in the heart is by percutaneous access and delivery using the Seldinger technique or other methods familiar to cardiologists. These approaches enable the pump 10 to be used in emergency medicine, a catheter lab, and in other non-surgical settings. Modifications can also enable the pump 10 to support the right side of the heart.

When a Seldinger insertion technique is used to advance the operative device to the heart, a guidewire and guidewire guide tube may be used. For example, the guidewire guide tube may be disposed through a central lumen of the catheter pump. The clinician can insert a guidewire through the guidewire guide tube, and can advance the guidewire to the heart. After advancing the operative device over the guidewire and into the heart, the guidewire and guidewire guide can be removed from the catheter pump. When the guidewire guide tube and/or the guidewire is retracted through a distal portion of a nose member, the distal portion may not adequately reseal the lumen. Accordingly, there is a need for an improved distal bearing support that provides for a re-sealable member and a nose member.

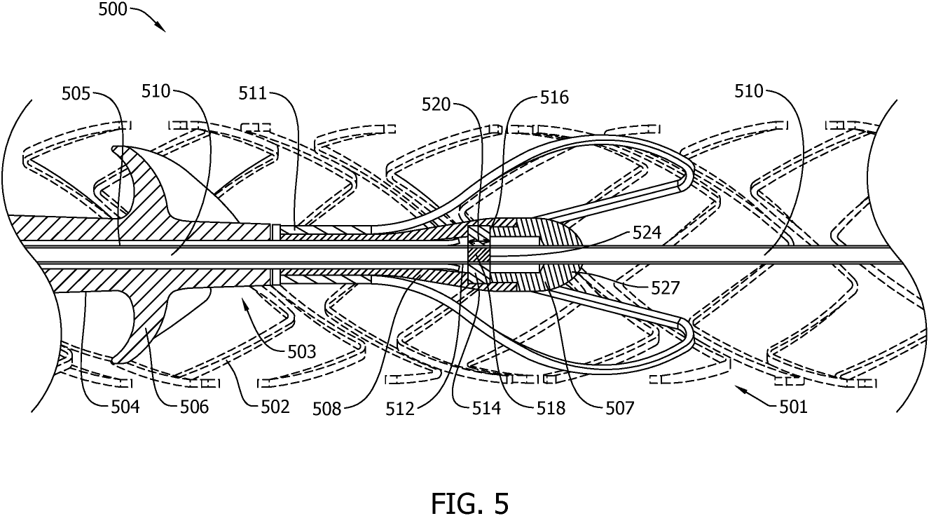

FIG. 5 shows aspects of one embodiment of an operative device 500 of a catheter pump, with a re-sealable member 514 and a nose member 507 of a distal bearing support 501. The operative device 500 can include a cannula housing 502, an impeller 503 disposed within the cannula housing 502, and the distal bearing support 501 configured to improve the bending stiffness and maneuverability of the operative device 500. The impeller 503 can include an impeller hub 504 mounted on an impeller shaft 505 and one or more blades 506 extending from the impeller hub 504. The distal bearing support 501 can include a nose member 507 configured to smooth the flow of blood. The distal bearing support 501 can also include a mounting portion 508 configured to mount to the impeller shaft 505, and a support member 509 (not shown) coupled to the mounting portion 508. A guidewire guide tube 510 can pass through the impeller shaft 505. A guidewire can be advanced through the guidewire guide tube 510 and into the patient’s anatomy. A proximal portion 511 of the support member 509 overlaps a distal end 512 of the impeller shaft 505, which can reduce the stiff length of the operative device 500.

A re-sealable member 514 can be inserted within a stepped region or recess near a distal end 516 of the mounting portion 508, e.g., into an enlarged portion disposed distal the enlarged portion in which the distal end 512 of the impeller shaft 505 is disposed. The re-sealable member 514 can be employed to reseal the aperture formed when the guidewire and/or guidewire guide tube 510 (e.g., made of stainless steel) is removed. In one embodiment, the re-sealable member 514 may be a septum (as shown in FIG. 5) and the mounting portion 508 may press against the re-sealable member 514 to compress or force the re-sealable member 514 radially inward, such that the re-sealable member 514 is pre-loaded to re-seal the lumen when the guidewire and/or the guidewire guide tube 510 is removed. The re-sealable member 514 may include a path 518 along a length dimension 520 of the re-sealable member 514 and an opening 522 (not shown) at a distal end 524 of the path 518 of the re-sealable member.

The nose member 507 may include an aperture 525 (not shown) at a distal portion 527 of the nose member 507. The aperture 525 (not shown) may be located distally of the opening 522 (not shown) of the re-sealable member 514, through which the guidewire and/or guidewire guide tube 510 can be passed when removing the guidewire and/or guidewire guide tube 510.

In some embodiments, the re-sealable member 514 may not rotate relative to the impeller shaft 505 and/or the mounting portion 508. In other embodiments, the re-sealable member 514 may rotate with the mounting portion 508. The re-sealable member 514 can be a self-healing polymer and/or a high durometer polymer, or any other polymer suitable for resealing after removal of the guidewire guide tube 510. In some embodiments, the re-sealable member 514 can be Nusil MED-4950, a silicone elastomer material to facilitate reduced material tearing. In other embodiments, the re-sealable member 514 can be a thermoset elastomer material to facilitate increased shape retention. As shown in FIG. 5, the re-sealable member 514 can be disposed distally of the impeller shaft 505 in the stepped region or recess of a distal portion of the mounting portion 508 (e.g., an interface member). In addition, the flared portion at the distal end 512 of the impeller shaft 505 can be disposed in or near the recess that includes the re-sealable member 514.

In some embodiments, one method of assembly of the path 518 through the re-sealable member 514 and the opening 522 at the distal end of the re-sealable member may be piercing the re-sealable member 514 after installation of the re-sealable member 514 into the stepped region or recess near the distal end 516 of mounting portion 508, e.g., into an enlarged portion disposed distal the enlarged portion in which the distal end 512 of the impeller shaft 505 is disposed. In other embodiments, another method of assembly of the path 518 through the re-sealable member 514 and the opening 522 at the distal end of the re-sealable member may be piercing the re-sealable member 514 prior to installation of the re-sealable member 514 into the stepped region or recess near the distal end 516 of mounting portion 508, e.g., into an enlarged portion disposed distal the enlarged portion in which the distal end 512 of the impeller shaft 505 is disposed.

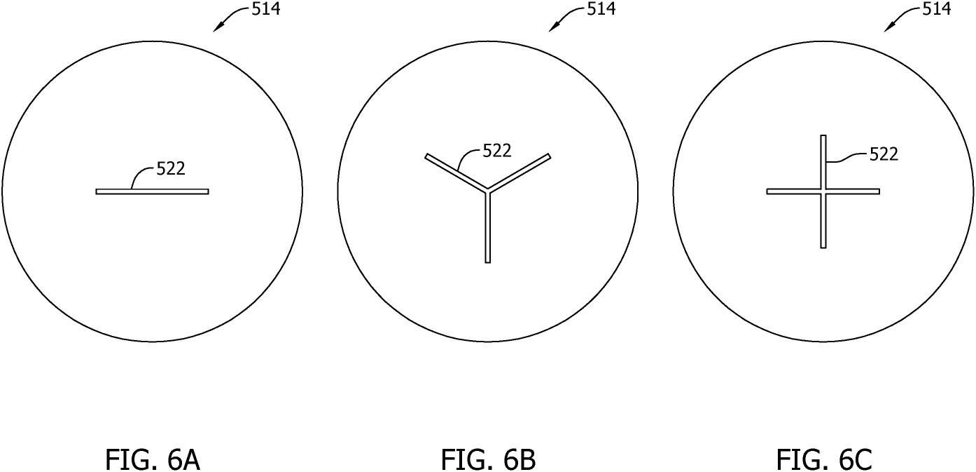

FIGS. 6A-6C show a top-view of different embodiments of an opening 522 in a re-sealable member 514 of a distal bearing support 501, as shown in FIG. 5. The opening 522 in the re-sealable member 514 can be of a varying size. Additionally, the opening 522 can be of a varying shape, as illustrated by FIGS. 6A-6C. The opening 522 can include a single slit (shown in FIG. 6A), three slits connected at one end and offset from each other at an angle slightly greater than 90° (shown in FIG. 6B), or two slits perpendicular and connected at the midpoint of each slit (shown in FIG. 6C).

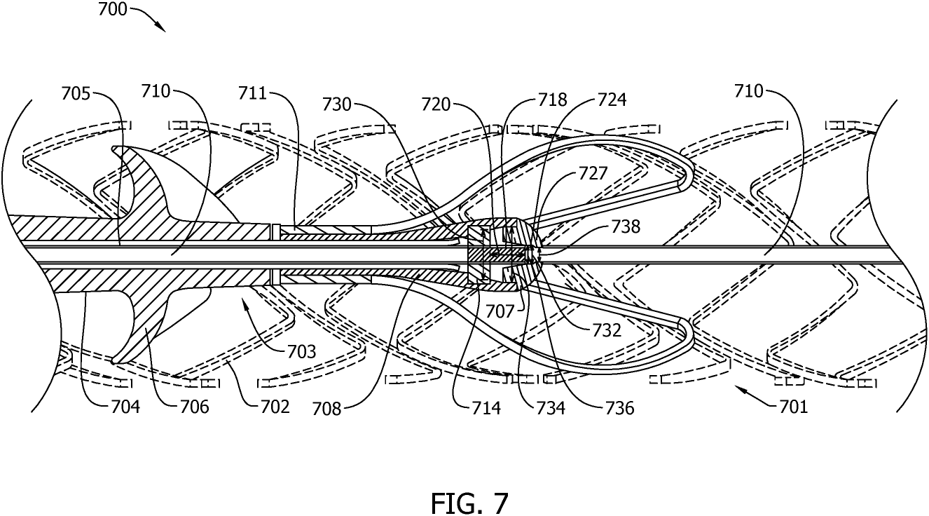

FIG. 7 shows aspects of another embodiment of an operative device 700 of a catheter pump, with a re-sealable member 714 and a nose member 707 of a distal bearing support 701. Unless otherwise noted, the reference numerals of FIG. 7 may refer to components similar to those referenced above in FIG. 5, incremented by 200 relative to FIG. 5. The re-sealable member 714 may include an increased length dimension 720, increasing the length of a path 718 through the re-sealable member 714 to facilitate the resealing of an opening 722 (not shown) at a distal end 726 of the re-sealable member 714. The increased length dimension 720 may be created by having a first diameter 730, a second diameter 732, and a third diameter 734, the first diameter 730 being greater than the second diameter 732, and the third diameter 734 being greater than the second diameter 732. Further, the increased length dimension 720 may be created by having a taper between the first diameter 730 and second diameter 732, and between the second diameter 732 and third diameter 734. In some embodiments, the re-sealable member 714 may be larger than the first diameter 730 and the second diameter 732. This may cause the re-sealable member 714 to be under compression.

The nose member 707 may include an aperture 725 (not shown) at a distal portion 727 of the nose member 707. The aperture 725 may include a first aperture diameter 708 and a second aperture diameter 738 distal of the first aperture diameter 708, the first aperture diameter 708 being greater than the second aperture diameter 738. In some embodiments, the first aperture diameter 708 may be equal to the third diameter 734.

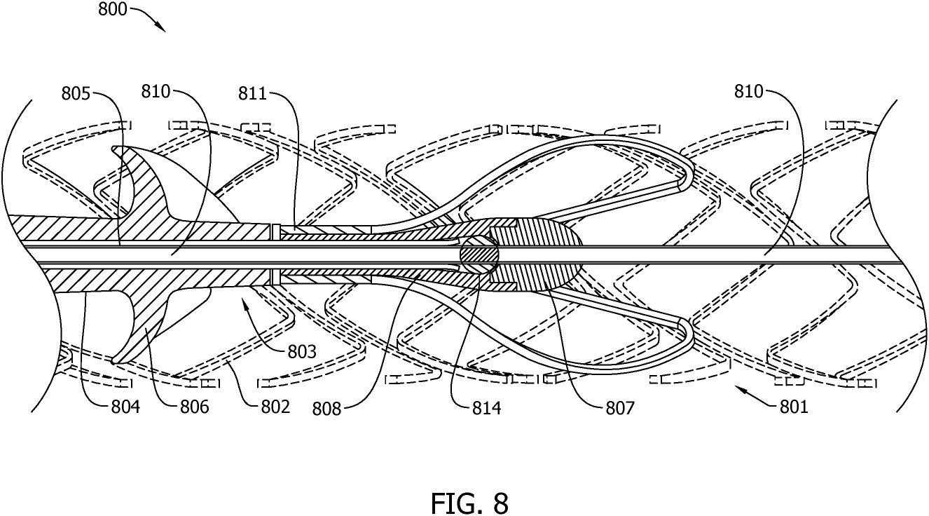

FIG. 8 shows aspects of yet another embodiment of an operative device 800 of a catheter pump, with a re-sealable member 814 and a nose member 807 of a distal bearing support 801. Unless otherwise noted, the reference numerals of FIG. 8 may refer to components similar to those referenced above in FIGS. 5 and 7, incremented by 100 relative to FIG. 7. The re-sealable member 814 may be a spherical shape. The enlarged distal portion of the cylindrical member may also be a spherical shape.

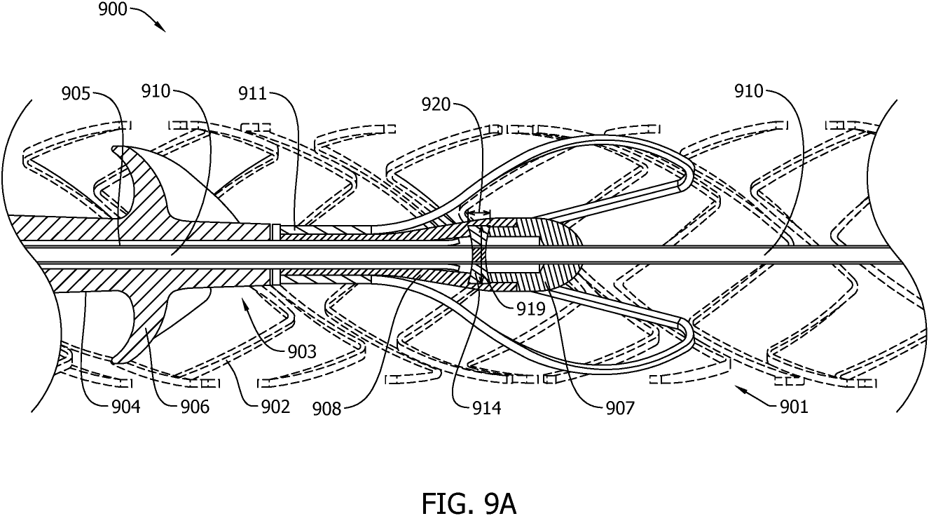

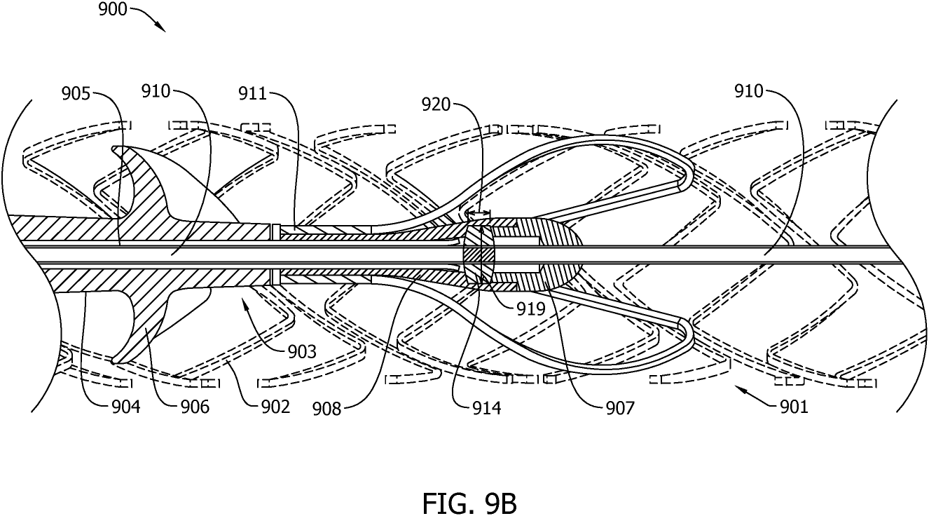

FIGS. 9A-9B show aspects of embodiments of an operative device 900 of a catheter pump, with varying shapes of a re-sealable member 914 of a distal bearing support 901. Unless otherwise noted, the reference numerals of FIG. 9 may refer to components similar to those referenced above in FIGS. 5, 7, and 8, incremented by 100 relative to FIG. 8. The re-sealable member 914 may include a width dimension 919 perpendicular to a length dimension 920. The re-sealable member 914 may be of a concave shape along the width dimension 919 (shown in FIG. 9A) or of a convex shape along the width dimension 919 (shown in FIG. 9B).

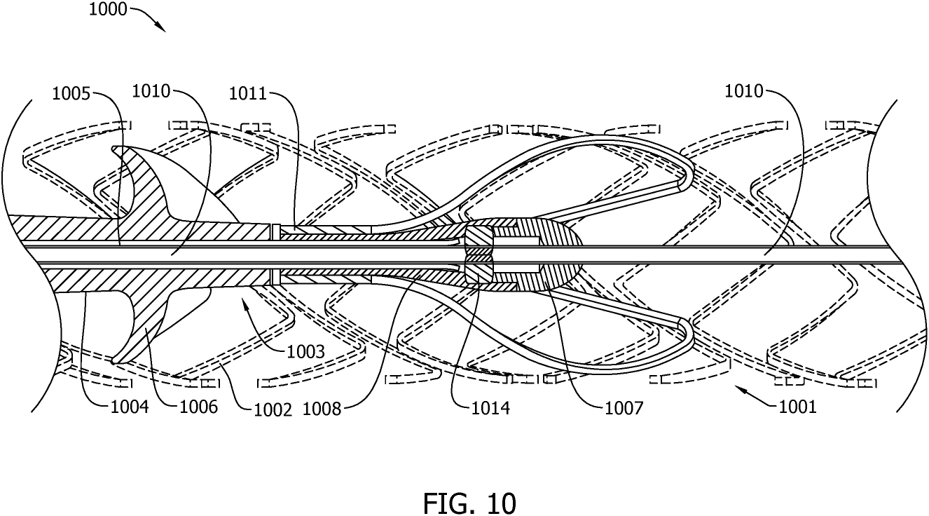

FIG. 10 shows aspects of another embodiment of an operative device 1000 of a catheter pump, with a re-sealable member 1014 of a distal bearing support 1001. Unless otherwise noted, the reference numerals of FIG. 10 may refer to components similar to those referenced above in FIGS. 5, 7, 8, and 9A-9B, incremented by 100 relative to FIGS. 9A-9B. The re-sealable member 1014 may be of a doughnut shape with a closed off hole. The re-sealable member 1014 may comprise an elastomer material in the doughnut shape.

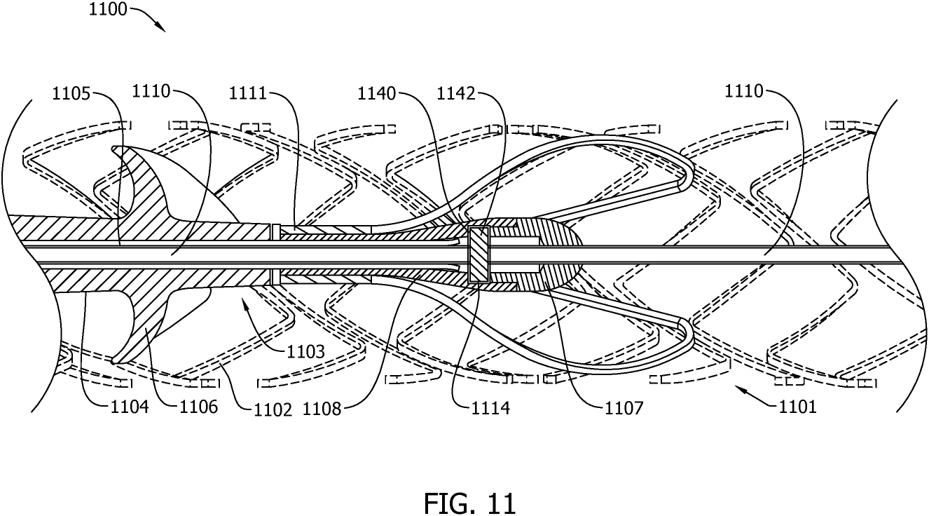

FIG. 11 shows aspects of yet another embodiment of an operative device 1100 of a catheter pump, with a re-sealable member 1114 of a distal bearing support 1101. Unless otherwise noted, the reference numerals of FIG. 11 may refer to components similar to those referenced above in FIGS. 5, 7, 8, 9A-9B, and 10, incremented by 100 relative to FIG. 10. The re-sealable member 1114 may be of a shell shape, including a shell 1140 and a shell filling 1142. The shell 1140 may comprise an elastomeric material, including silicone, polyurethane, Viton, fluoroelastomer, nitrile rubber, styrene butadiene rubber (SBR), neoprene, ethylene propylene diene monomer (EPDM), poly(styrene-block-isoButylene-block-styrene) (SIBS), or latex. The shell filling 1142 may comprise a high viscosity gel, including a hydrophobic gel or an aqueous based gel.

In the implementation of FIGS. 5, 7, 8, 9A-9B, 10, and 11, the stiff length may be reduced while also introducing a re-sealable member to prevent fluid from entering the apertures formed when the guidewire guide tube is retracted after using a guidewire with the Seldinger technique.

Modifications of catheter pumps incorporating a catheter assembly with a distal impeller support can be used for right side support. For example, a catheter body carrying an impeller and distal bearing support can be formed to have a deployed shape corresponding to the shape of the vasculature traversed between a peripheral vascular access point and the right ventricle. One will appreciate from the description herein that the catheter assembly may be modified based on the respective anatomy to suit the desired vascular approach. For example, the catheter assembly in the insertion state may be shaped for introduction through the subclavian artery to the heart. The catheter pump may be configured for insertion through a smaller opening and with a lower average flow rate for right side support. In various embodiments, the catheter assembly is scaled up for a higher flow rate for sicker patients and/or larger patients.

Although the inventions herein have been described with reference to particular embodiments, it is to be understood that these embodiments are merely illustrative of the principles and applications of the present inventions. It is therefore to be understood that numerous modifications can be made to the illustrative embodiments and that other arrangements can be devised without departing from the spirit and scope of the present inventions as defined by the appended claims. Thus, it is intended that the present application cover the modifications and variations of these embodiments and their equivalents.

What is claimed is:

1. A catheter pump, comprising:
an elongated catheter body having a distal portion including an expandable cannula having an inlet and an outlet, the expandable cannula having a delivery profile and an operational profile larger than the delivery profile;
an impeller assembly including an impeller shaft and an impeller body including one or more blades, the one or more blades drawing blood into the expandable cannula when rotated; and
an expandable support having a mounting portion disposed on the impeller shaft distal of the impeller body and configured to maintain a position of the impeller assembly relative to a cannula wall, the mounting portion comprising:
a cylindrical member disposed on the impeller shaft, the cylindrical member comprising an enlarged distal portion having an inner diameter greater than an enlarged diameter at a distal end of the impeller shaft and a nose member; and
a re-sealable member disposed in the enlarged distal portion of the cylindrical member, the re-sealable member comprising a path through the re-sealable member along a length dimension of the re-sealable member and an opening at a distal end of the path through which a guidewire can be positioned,
wherein the nose member is coupled with the distal portion of the cylindrical member, the nose member comprising a distal portion with an aperture distal of the re-sealable member for passage of the guidewire.

2. The catheter pump of claim 1, wherein the re-sealable member comprises a septum.

3. The catheter pump of claim 1, wherein the re-sealable member reseals the opening at the distal end of the path through the re-sealable member when the guidewire is removed.

4. The catheter pump of claim 2, further comprising a plurality of re-sealable members, the plurality of re-sealable members comprising a distal re-sealable member disposed in the enlarged distal portion of the cylindrical member and a proximal re-sealable member disposed in a proximal portion of the elongated catheter body.

5. The catheter pump of claim 2, wherein the path and opening at the distal end of the path is created prior to installation of the re-sealable member within the enlarged distal portion of the cylindrical member.

6. The catheter pump of claim 5, wherein the opening at the distal end of the path comprises a single slit.

7. The catheter pump of claim 5, wherein the opening at the distal end of the path comprises three slits, the slits connected at one end and offset by an angle slightly greater than 90°.

8. The catheter pump of claim 5, wherein the opening at the distal end of the path comprises two slits, the slits perpendicular and connected at a midpoint of each slit.

9. The catheter pump of claim 2, wherein the path through the re-sealable member is along an increased length dimension of the re-sealable member.

10. The catheter pump of claim 9, wherein the enlarged distal portion of the cylindrical member comprises a first diameter, a second diameter, a third diameter, a first taper, and a second taper, the first taper between the first and second diameter and the second taper between the second and third diameter, the second diameter being greater than the first diameter and the third diameter, and the first diameter being greater than the third diameter.

11. The catheter pump of claim 10, wherein the distal portion of the nose member comprises a first aperture diameter and a second aperture diameter distal of the first aperture diameter, the first aperture diameter being greater than the second aperture diameter.

12. The catheter pump of claim 2, wherein the re-sealable member is of a spherical shape.

13. The catheter pump of claim 12, wherein the enlarged distal portion of the cylindrical member is of a spherical shape.

14. The catheter pump of claim 2, wherein the re-sealable member comprises a width dimension perpendicular to the length dimension, the re-sealable member being of a concave shape along the width dimension.

15. The catheter pump of claim 2, wherein the re-sealable member is of a convex shape along a width dimension.

16. The catheter pump of claim 2, wherein the re-sealable member comprises an elastomeric shell filled with a flowable gel.

17. The catheter pump of claim 2, wherein the re-sealable member comprises a thermoset elastomer.

18. The catheter pump of claim 2, wherein the re-sealable member comprises a higher durometer silicone.

19. The catheter pump of claim 1, wherein the re-sealable member comprises an elastomer doughnut, the elastomer doughnut comprising a closed off hole.

20. The catheter pump of claim 1, wherein the elongated catheter body comprises a proximal portion with an anti-foaming agent.

21. An apparatus for inducing motion of a fluid relative to the apparatus, comprising:
a motor;
an elongated catheter body coupled with the motor, the elongated catheter body including an expandable distal portion having an inlet and an outlet and a support structure disposed about a lumen, the expandable distal portion having a delivery profile and an operational profile larger than the delivery profile;
an impeller comprising at least one impeller blade;
an expandable impeller support having an arcuate outer surface in contact with the support structure at least when the expandable distal portion has the operational profile;
a re-sealable member disposed in an expandable distal portion of the support structure distally of the impeller, the re-sealable member comprising a path through the re-sealable member along a length dimension of the re-sealable member and an opening at a distal end of the path through which a guidewire can be positioned; and
a nose member disposed distally of the re-sealable member, the nose member comprising a distal portion with an aperture distal of the re-sealable member for passage of the guidewire,
wherein operation of the motor causes rotation of the impeller to draw blood into the lumen.

22. The apparatus of claim 21, wherein the re-sealable member comprises a septum.

23. The apparatus of claim 22, wherein the re-sealable member reseals the opening at the distal end of the path through the re-sealable member when the guidewire is removed.

24. The apparatus of claim 23, further comprising a plurality of re-sealable members, the plurality of re-sealable members comprising a distal re-sealable member disposed in the expandable distal portion of the support structure and a proximal re-sealable member disposed in a proximal portion of the elongated catheter body.

25. The apparatus of claim 23, wherein the path and opening at the distal end of the path is created prior to installation of the re-sealable member within the expandable distal portion of the support structure.

26. The apparatus of claim 21, wherein the opening at the distal end of the path comprises a single slit.

27. The apparatus of claim 26, wherein the opening at the distal end of the path comprises three slits, the slits connected at one end and offset by an angle slightly greater than 90°.

28. The apparatus of claim 26, wherein the opening at the distal end of the path comprises two slits, the slits perpendicular and connected at a midpoint of each slit.

29. The apparatus of claim 23, wherein the path through the re-sealable member is along an increased length dimension of the re-sealable member.

30. The apparatus of claim 29, wherein the expandable distal portion of the support structure comprises a first diameter, a second diameter, a third diameter, a first taper, and a second taper, the first taper between the first and second diameter and the second taper between the second and third diameter, the second diameter being greater than the first diameter and the third diameter and the first diameter being greater than the third diameter.

31. The apparatus of claim 30, wherein the distal portion of the nose member comprises a first aperture diameter and a second aperture diameter distal of the first aperture diameter, the first aperture diameter being greater than the second aperture diameter.

32. The apparatus of claim 23, wherein the re-sealable member is of a spherical shape.

33. The apparatus of claim 23, wherein the distal portion of the nose member is rounded.

34. The apparatus of claim 23, wherein the re-sealable member comprises a width dimension perpendicular to the length dimension, the re-sealable member being of a concave shape along the width dimension.

35. The apparatus of claim 23, wherein the re-sealable member is of a convex shape along a width dimension.

36. The apparatus of claim 23, wherein the re-sealable member comprises an elastomeric shell filled with a flowable gel.

37. The apparatus of claim 23, wherein the re-sealable member comprises a thermoset elastomer.

38. The apparatus of claim 23, wherein the re-sealable member comprises a higher durometer silicone.

39. The apparatus of claim 22, wherein the re-sealable member comprises an elastomer doughnut, the elastomer doughnut comprising a closed off hole.

40. The apparatus of claim 22, wherein the elongated catheter body comprises a proximal portion with an anti-foaming agent.
